(19) United States
(12) Patent Application Publication
(10) Pub. No.: US 2026/0317256 A1
(43) Pub. Date: Oct. 15, 2026

(54) METHOD AND APPARATUS FOR SYNTHESIZING CF4 BY ACTIVATING SF6 USING TWO-STAGE DIELECTRIC BARRIER DISCHARGE

(71) Applicants: Hubei University Of Technology(hbut), Wuhan (CN); CSG Electric Power Research Institute Co, Ltd., Guangzhou (CN)

(72) Inventors: Xiaoxing ZHANG, Wuhan (CN); Yalong LI, Wuhan (CN); Mingli FU, Wuhan (CN); Zhe HUANG, Wuhan (CN); Yangjing LE, Wuhan (CN); Zhaodi YANG, Wuhan (CN); Chao GAO, Wuhan (CN); Yan LUO, Wuhan (CN); Lei YU, Wuhan (CN); Huiyu TAN, Wuhan (CN)

(73) Assignees: Hubei University Of Technology(hbut), Wuhan (CN); CSG Electric Power Research Institute Co., Ltd., Guangzhou (CN)

(21) Appl. No.: 19/363,698

(22) Filed: Oct. 21, 2025

(30) Foreign Application Priority Data
Jan. 13, 2025 (CN) 2025100485119

(51) Int. Cl. C07C 17/23; B01J 8/02; H05H 1/24

(52) U.S. Cl. CPC C07C 17/23; B01J 8/0278; B01J 2208/00946; H05H 1/2406; H05H 2245/10

(57) ABSTRACT

Disclosed are a method and apparatus for synthesizing CF₄ by activating SF₆ using two-stage dielectric barrier discharge. A method for synthesizing CF₄ by activating and degrading SF₆ using a two-stage DBD plasma and introducing CO₂ gas is proposed, where a first-stage DBD plasma reactor degrades SF₆ molecules, decomposing them into fluorine atoms and other low-fluorine sulfur small molecules, which not only achieves effective degradation of SF₆ but also provides a necessary fluorine source for subsequent CF₄ synthesis; a second-stage DBD plasma reactor uses CO₂ as a carbon source, which reacts with the low-fluorine sulfur small molecules produced in the first stage to generate CF₄ gas, where a mixed gas containing the CF₄ gas undergoes primary condensation to condense some fluorine-containing acidic waste gases into a liquid, and subsequently the remaining mixed gas undergoes secondary condensation to condense the CF₄ gas into a liquid for collection.

### CROSS-REFERENCE TO RELATED APPLICATIONS

The application claims priority to Chinese patent application No. 2025100485119, filed on Jan. 13, 2025, the entire contents of which are incorporated herein by reference.

### TECHNICAL FIELD

This disclosure relates to the technical field of efficient degradation and resource utilization of sulfur hexafluoride and, in particular, to a method and apparatus for synthesizing CF₄ by activating SF₆ using two-stage dielectric barrier discharge.

### BACKGROUND

Sulfur hexafluoride (SF₆), as a colorless, odorless, non-toxic, and non-flammable gas, is a synthetic fluoride known for its excellent electrical insulation and arc-quenching properties, commonly used in electrical insulation equipment. In recent years, with the rapid development of the power industry, the usage of SF₆ has been increasing annually. However, equipment maintenance and replacement in power systems lead to substantial emissions of SF₆ gas every year. Due to its octahedral molecular structure, which is extremely stable, SF₆ is difficult to degrade naturally in the atmosphere. Furthermore, as a potent greenhouse gas, its global warming potential is 23,500 times that of carbon dioxide (CO₂). With the introduction of carbon peaking and carbon neutrality policies, the degradation treatment of SF₆ has become imperative.

Currently, the degradation treatment of SF₆ primarily includes: thermal catalytic degradation, photocatalytic degradation, and plasma degradation, where the plasma degradation utilizes effective active species such as energetic electrons and active particles in the plasma to collide with SF₆ gas molecules, causing their dissociation in a short time. Depending on the method of plasma generation, it can be classified into radio frequency discharge, electron beam discharge, microwave discharge, and dielectric barrier discharge plasma, where the dielectric barrier discharge plasma utilizes continuous discharge breakdown and blocking between electrodes and insulating dielectrics to generate a large number of active species. As a type of low-temperature plasma, it offers numerous advantages such as convenient and flexible setup, mild reaction conditions, ease of control, high energy efficiency, and safe and reliable operation, making it widely used in waste gas treatment.

In recent years, carbon tetrafluoride (CF₄) has been widely used in various electronic industries, such as semiconductor manufacturing and refrigeration. Conventional methods for synthesizing CF₄ generally suffer from harsh reaction conditions, low efficiency, high energy consumption, and high costs. Currently, the primary methods for preparing CF₄ include direct fluorination of alkanes, direct carbon-fluorine synthesis, and fluorination of hydrofluoromethane, where the direct fluorination of alkanes is the earliest industrial method for preparing fluoroalkanes, which is relatively mature and operationally simple, but the reaction process is difficult to control, resulting in complex products and low yield; the direct carbon-fluorine synthesis involves the reaction of carbon with fluorine gas and, although it offers high yield, poses great safety risks and requires some special measures to control the reaction conditions; the fluorination of hydrofluoromethane for synthesizing CF₄ has a simple process but high synthesis costs, making it difficult to scale up for application. The aforementioned methods are plagued by harsh reaction conditions, low yield, high costs, and safety concerns. Currently, there is an urgent need for a green synthesis technology that is safe and reliable, features mild reaction conditions, offers high yield and efficiency, and has lower costs to overcome these challenges.

### SUMMARY

Based on the prior art mentioned above, this disclosure provides a method and apparatus for synthesizing CF₄ by activating SF₆ using two-stage dielectric barrier discharge, which can not only simultaneously degrade two greenhouse gases, CO₂ and SF₆, reducing the hazards of the greenhouse effect, but also synthesize the electronic specialty gas CF₄, thereby achieving the fluorine resource utilization of SF₆ and the preparation of high-value CF₄ gas. This disclosure can provide a new approach for the resource utilization of SF₆, the treatment of CO₂, and the synthesis of the electronic specialty gas CF₄.

The technical solutions for achieving the above objectives of this disclosure are as follows:

a method for synthesizing CF₄ by activating SF₆ using two-stage dielectric barrier discharge (DBD), including the following steps:
S1: introducing a background gas and a sulfur hexafluoride gas into a first-stage DBD plasma reactor, where the background gas is ionized to generate plasma, the sulfur hexafluoride, after being activated by the plasma, is ionized into fluorine atoms and low-fluorine sulfides, and the generated fluorine atoms and low-fluorine sulfides further react with oxygen and silicon dioxide to generate fluorine-containing small molecules; and
S2: subsequently introducing the fluorine-containing small molecules generated in the step S1 into a second-stage DBD plasma reactor connected to the first-stage DBD plasma reactor, and simultaneously introducing carbon dioxide gas into the second-stage DBD plasma reactor, where the low-fluorine sulfides react with the carbon dioxide under an action of the plasma to generate carbon tetrafluoride gas.

Further, the background gas is an inert gas.

Further, in the step S1, a discharge region within the first-stage DBD plasma reactor is filled with a catalyst γ-Al₂O₃.

Further, the step S2 includes: first performing primary condensation on a mixed gas containing carbon tetrafluoride flowing out from an outlet of the second-stage DBD plasma reactor to condense fluorine-containing acidic waste gases into a liquid, then performing secondary condensation on the remaining mixed gas to condense the generated carbon tetrafluoride gas into a liquid, and collecting the liquid carbon tetrafluoride.

Further, the primary condensation is performed at −60° C. to −50° C., and the secondary condensation is performed at −150° C. to −130° C.

An apparatus for synthesizing CF₄ by activating SF₆ using two-stage DBD, including a sulfur hexafluoride gas supply unit, a background gas supply unit, a first-stage DBD plasma reactor, a second-stage DBD plasma reactor, a carbon dioxide gas supply unit, a primary condensation unit, and a secondary condensation unit, where the sulfur hexafluoride gas supply unit and the background gas supply unit are respectively in communication with an inlet of the first-stage DBD plasma reactor, an outlet of the first-stage DBD plasma reactor and the carbon dioxide gas supply unit are respectively connected to an inlet of the second-stage DBD plasma reactor, an outlet of the second-stage DBD plasma reactor is connected to an inlet of the primary condensation unit, and a gas outlet of the primary condensation unit is connected to an inlet of the secondary condensation unit.

The apparatus further includes a gas mixer, where the sulfur hexafluoride gas supply unit includes a sulfur hexafluoride gas cylinder, a first gas supply branch pipe, and a first pressure reducing valve, where an upstream end of the first gas supply branch pipe is connected to the sulfur hexafluoride gas cylinder, and the first pressure reducing valve is installed on the first gas supply branch pipe; the background gas supply unit includes a background gas cylinder, a second gas supply branch pipe, and a second pressure reducing valve, where an upstream end of the second gas supply branch pipe is connected to the background gas cylinder, the second pressure reducing valve is installed on the second gas supply branch pipe, downstream ends of the first gas supply branch pipe and the second gas supply branch pipe are respectively connected to the gas mixer, and an outlet of the gas mixer is connected to the inlet of the first-stage DBD plasma reactor.

The carbon dioxide gas supply unit includes a carbon dioxide gas cylinder, a third gas supply branch pipe, and a third pressure reducing valve, where an upstream end of the third gas supply branch pipe is connected to the carbon dioxide gas cylinder, the third pressure reducing valve is installed on the third gas supply branch pipe, and a downstream end of the third gas supply branch pipe is connected to the inlet of the second-stage DBD plasma reactor.

The primary condensation unit includes a primary condenser, where the primary condenser includes a first gas outlet and a first liquid outlet; the secondary condensation unit includes a secondary condenser, where the secondary condenser includes a second gas outlet and a second liquid outlet; the outlet of the second-stage DBD plasma reactor is connected to an inlet of the primary condenser; and the first gas outlet is connected to an inlet of the secondary condenser.

The secondary condensation unit further includes a CF₄ collection tank, and the second liquid outlet is connected to the CF₄ collection tank.

A condensing medium in the primary condenser is an ethylene glycol aqueous solution, and a condensing medium in the secondary condenser is liquid nitrogen.

The apparatus further includes an exhaust gas treatment unit, where the exhaust gas treatment unit includes an alkali liquor treatment tank and an exhaust gas pipe, and both ends of the exhaust gas pipe are respectively connected to the alkali liquor treatment tank and the second gas outlet.

Compared with the prior art, this disclosure has the following advantages and beneficial effects:
1: this disclosure proposes a method for synthesizing CF₄ by activating and degrading SF₆ using a two-stage DBD plasma and introducing CO₂ gas, where a first-stage DBD plasma reactor degrades SF₆ molecules, decomposing them into fluorine atoms and other low-fluorine sulfur small molecules, which not only achieves effective degradation of SF₆ but also provides a necessary fluorine source for subsequent CF₄ synthesis; a second-stage DBD plasma reactor uses CO₂ as a carbon source, which reacts with the fluorine-containing small molecules produced in the first stage to generate CF₄ gas, where a mixed gas containing the CF₄ gas undergoes primary condensation to condense small molecules of some fluorine-containing acidic waste gases into a liquid, and subsequently the remaining mixed gas undergoes secondary condensation to condense the CF₄ gas into a liquid for collection, achieving efficient separation and purification of the product. Some acidic gases that do not participate in the reaction are treated by an alkali liquor treatment tank, which can reduce harm to the environment;
2: by adopting the two-stage DBD reaction structure, this disclosure not only achieves high-efficiency degradation of SF₆ but also significantly increases the synthesis rate of CF₄. This disclosure can not only solve the limitations and shortcomings existing in conventional CF₄ preparation methods but also provide a new approach for the fluorine resource utilization of SF₆, the treatment of CO₂ as a greenhouse gas, and the synthesis of high-value chemicals;
3: the method of this disclosure can not only achieve complete degradation of SF₆ but also use SF₆ as a fluorine source and CO₂ as a carbon source to react under an action of plasma to synthesize a high-value electronic specialty gas CF₄ used in semiconductor manufacturing, thereby solving the problem of difficulty in treating SF₆ while utilizing its fluorine resource in combination with CO₂ to synthesize CF₄, turning waste into treasure;
4: this disclosure employs the two-stage DBD reaction structure, in which two reaction devices have different discharge conditions and can discharge independently. This staged treatment method helps to control the reaction process more effectively and improve the purity and yield of the product; and
5: by filling the first-stage plasma reactor with γ-Al₂O₃, this disclosure enables complete degradation of SF₆, allowing more low-fluorine sulfides to react with CO₂ in the second-stage reaction tube to generate CF₄.

### BRIEF DESCRIPTION OF THE DRAWINGS

FIG. 1 is a schematic structural diagram of an apparatus for synthesizing CF₄ by activating SF₆ using two-stage dielectric barrier discharge;

FIG. 2 shows a gas chromatogram of a mixed gas at an outlet of a second-stage DBD plasma reactor in Example 2; and

FIG. 3 shows a gas chromatogram of a mixed gas at an outlet of a first-stage DBD plasma reactor in Example 3.

Reference signs used in the figures: 1—sulfur hexafluoride gas cylinder; 2—first gas supply branch pipe; 3—first pressure reducing valve; 4—argon gas cylinder; 5—second gas supply branch pipe; 6—second pressure reducing valve; 7—gas mixer; 8—carbon dioxide gas cylinder; 9—third gas supply branch pipe; 10—third pressure reducing valve; 11—first-stage DBD plasma reactor; 12—second-stage DBD plasma reactor; 13—primary condenser: 1301—first gas outlet, 1302—first liquid outlet; 14—liquid waste tank; 15—secondary condenser: 1501—second gas outlet, 1502—second liquid outlet; 16—CF₄ collection tank; 17—alkali liquor treatment tank; and 18—exhaust gas pipe.

### DETAILED DESCRIPTION OF THE EMBODIMENTS

An apparatus for synthesizing CF₄ by activating SF₆ using two-stage dielectric barrier discharge according to this disclosure will be described in detail below with reference to the accompanying drawings.

### Example 1

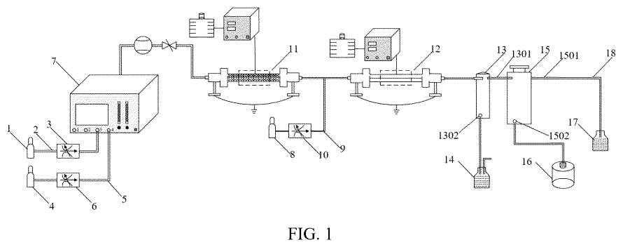

An apparatus for synthesizing CF₄ by activating SF₆ using two-stage dielectric barrier discharge provided in this example has a structure shown in FIG. 1, including a sulfur hexafluoride gas supply unit, an argon gas supply unit, a gas mixer 7, a first-stage DBD plasma reactor 11, a second-stage DBD plasma reactor 12, a carbon dioxide gas supply unit, a primary condensation unit, a secondary condensation unit, and an exhaust gas treatment unit.

The sulfur hexafluoride gas supply unit includes a sulfur hexafluoride gas cylinder 1, a first gas supply branch pipe 2, and a first pressure reducing valve 3, where an upstream end of the first gas supply branch pipe 2 is connected to the sulfur hexafluoride gas cylinder 1, and the first pressure reducing valve 3 is installed on the first gas supply branch pipe 2.

The background gas supply unit includes an argon gas cylinder 4, a second gas supply branch pipe 5, and a second pressure reducing valve 6, where an upstream end of the second gas supply branch pipe 5 is connected to the background gas cylinder 4, and the second pressure reducing valve 6 is installed on the second gas supply branch pipe 5.

Downstream ends of the first gas supply branch pipe 2 and the second gas supply branch pipe 5 are respectively connected to the gas mixer 7, and sulfur hexafluoride gas or sulfur hexafluoride waste gas enters the gas mixer 7 and is mixed uniformly therein.

The first-stage DBD plasma reactor 11 includes a coaxial DBD reaction tube, a plasma power supply, and a voltage regulator, where reaction conditions and parameters are controlled via the voltage regulator and the plasma power supply. An outlet of the gas mixer 7 is connected to an inlet of the coaxial DBD reaction tube of the first-stage DBD plasma reactor 11 via a pipeline, and an electromagnetic flowmeter and an electromagnetic valve are arranged on the pipeline connecting the outlet of the gas mixer 7 to the first-stage DBD plasma reactor 11.

The second-stage DBD plasma reactor 12 includes a coaxial DBD reaction tube, a plasma power supply, and a voltage regulator, where reaction conditions and parameters are controlled via the voltage regulator and the plasma power supply. An outlet of the coaxial DBD reaction tube of the first-stage DBD plasma reactor 11 is connected to an inlet of the coaxial DBD reaction tube of the second-stage DBD plasma reactor 12 via a pipeline.

The carbon dioxide gas supply unit includes a carbon dioxide gas cylinder 8, a third gas supply branch pipe 9, and a third pressure reducing valve 10, where an upstream end of the third gas supply branch pipe 9 is connected to the carbon dioxide gas cylinder 8, and the third pressure reducing valve 10 is installed on the third gas supply branch pipe 9. A downstream end of the third gas supply branch pipe 9 is connected to the pipeline connecting the outlet of the coaxial DBD reaction tube of the first-stage DBD plasma reactor 11 to the inlet of the coaxial DBD reaction tube of the second-stage DBD plasma reactor 12, and carbon dioxide is introduced into the coaxial DBD reaction tube of the second-stage DBD plasma reactor 12 from the carbon dioxide gas cylinder 8.

The primary condensation unit includes a primary condenser 13 and a liquid waste tank 14, where a condensing medium filled in the primary condenser 13 is an ethylene glycol aqueous solution, and the primary condenser 13 has a condensation temperature of −60° C. The primary condenser 13 includes a first gas outlet 1301 and a first liquid outlet 1302, where an outlet of the coaxial DBD reaction tube of the second-stage DBD plasma reactor 12 is connected to an inlet of the primary condenser 13 via a pipeline. The first liquid outlet 1302 is connected to the liquid waste tank 14 via a pipeline, and the liquid waste tank 14 is used for collecting liquefied fluorine-containing acidic waste gases.

The secondary condensation unit includes a secondary condenser 15 and a CF₄ collection tank 16, where a condensing medium filled in the secondary condenser 15 is liquid nitrogen, and the secondary condenser 15 has a condensation temperature of −150° C. The secondary condenser 15 includes a second gas outlet 1501 and a second liquid outlet 1502, where the first gas outlet 1301 is connected to an inlet of the secondary condenser 15. The second liquid outlet 1502 is connected to the CF₄ collection tank 16 via a pipeline, and the CF₄ collection tank 16 is used for collecting liquefied carbon tetrafluoride.

The exhaust gas treatment unit includes an alkali liquor treatment tank 17 and an exhaust gas pipe 18, where both ends of the exhaust gas pipe 18 are respectively connected to the alkali liquor treatment tank 17 and the second gas outlet 1501. The alkali liquor treatment tank 17 is provided with calcium hydroxide (Ca(OH)₂) alkali liquor therein to fully neutralize and absorb some unreacted acidic gases, thereby avoiding harm to the environment.

A method for synthesizing CF₄ by activating SF₆ using two-stage dielectric barrier discharge according to this disclosure will be described in detail below in conjunction with the above-described apparatus.

### Example 2

1: a discharge region of the coaxial DBD reaction tube of the first-stage DBD plasma reactor 11 was filled with a catalyst γ-Al₂O₃; the condensing medium, ethylene glycol aqueous solution, was added into the primary condenser 13 at a condensation temperature of −60° C.; and the condensing medium, liquid nitrogen, was added into the secondary condenser 15 at a condensation temperature of −150° C.;
2: the apparatus for synthesizing CF₄ by activating SF₆ using two-stage dielectric barrier discharge was assembled and connected according to the connection relationships of the above-described apparatus (as shown in FIG. 1);
3: the argon gas cylinder 4, the second pressure reducing valve 6, and the gas mixer 7 were opened; a valve on the exhaust gas pipe 18 was closed; argon gas was introduced to check the air tightness of the apparatus to prevent toxic gas leakage during the reaction from causing harm to personnel and to ensure the reaction proceeds stably and orderly; and after the check was completed, the argon gas cylinder 4, the second pressure reducing valve 6, and the gas mixer 7 were closed;
4: the input power of the first-stage DBD plasma reactor 11 was set to 100 W, and the input power of the second-stage DBD plasma reactor 12 was set to 100 W; the first-stage DBD plasma reactor 11 and the second-stage DBD plasma reactor 12 were started; the temperatures inside the coaxial DBD reaction tubes of both the first-stage DBD plasma reactor 11 and the second-stage DBD plasma reactor 12 began to rise, respectively; and after a period of time, the temperatures inside the coaxial DBD reaction tubes of both the first-stage DBD plasma reactor 11 and the second-stage DBD plasma reactor 12 stabilized at 105° C. and 115° C., respectively;
5: the sulfur hexafluoride gas cylinder 1, the first pressure reducing valve 3, the argon gas cylinder 4, the second pressure reducing valve 6, the valves both on the pipeline connecting the gas mixer 7 to the inlet of the first-stage DBD plasma reactor 11 and on the pipeline connecting the outlet of the first-stage DBD plasma reactor 11 to the inlet of the second-stage DBD plasma reactor 12, and the valve on the exhaust gas pipe 18 were opened; the sulfur hexafluoride gas from the sulfur hexafluoride gas cylinder 1 and the argon gas from the argon gas cylinder 4 entered the gas mixer 7, where they were mixed uniformly, and subsequently entered the coaxial DBD reaction tube of the first-stage DBD plasma reactor 11. The amounts of introduced sulfur hexafluoride gas and argon gas were precisely controlled via the electromagnetic flowmeter and electromagnetic valve connected to the outlet of the gas mixer 7, thereby controlling the concentration of SF₆ introduced into the first-stage DBD plasma reactor 11.

In this example, the flow rate of the sulfur hexafluoride gas was controlled to be 6 mL/min, the flow rate of the argon gas was controlled to be 294 mL/min, and the concentration of SF₆ was 2%. The SF₆ and argon gas were directly connected to two channels of a gas proportioner, forming a mixed gas inside the gas mixer 7. The output flow rate of the mixed gas was 300 mL/min, which was precisely controlled by the electromagnetic flowmeter and then fed into the first-stage DBD plasma reactor;
6: the argon gas was ionized to generate plasma in the coaxial DBD reaction tube of the first-stage DBD plasma reactor 11; the sulfur hexafluoride gas, after being activated by the plasma, was ionized into fluorine atoms and low-fluorine sulfides (such as SF₅ and SF₄); the generated fluorine atoms and low-fluorine sulfides further reacted with oxygen and silicon dioxide to form fluorine-containing small molecules; and these fluorine-containing small molecules entered the coaxial DBD reaction tube of the second-stage DBD plasma reactor 12;
7: after the first-stage DBD plasma reactor 11 had discharged for 15 min, the carbon dioxide gas cylinder 8 and the third pressure reducing valve 10 were opened; carbon dioxide entered the coaxial DBD reaction tube of the second-stage DBD plasma reactor 12 and reacted with the fluorine-containing small molecules in the coaxial DBD reaction tube of the second-stage DBD plasma reactor 12 to generate CF₄ gas.

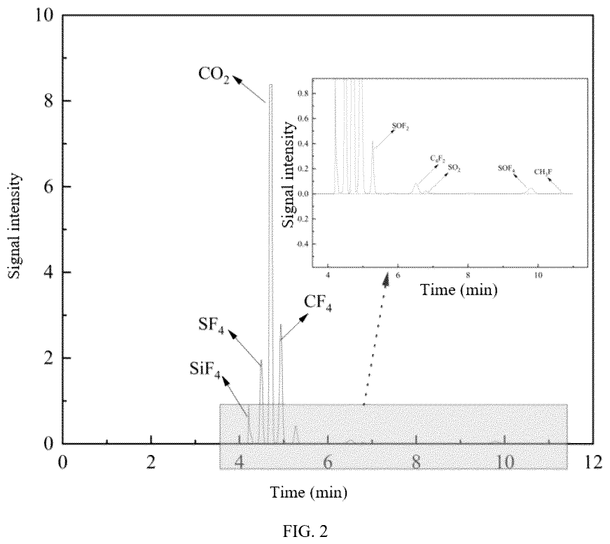

The mixed gas discharged from the outlet of the second-stage DBD plasma reactor was tested by a gas chromatograph-mass spectrometer. The obtained gas chromatogram is shown in FIG. 2. It can be seen from FIG. 2 that the CF₄ gas indeed existed in the mixed gas. Besides the CF₄ gas, there were also SOF₂, SOF₄, C₄F₂, SO₂, CH₃F, SiF₄, and SF₄;
8: the generated CF₄ gas and other gaseous products (such as SiF₄, SOF₂, SOF₄, and SO₂) formed a mixed gas that entered the primary condenser for condensation; some fluorine-containing acidic waste gases (such as SOF₂ and SOF₄) were condensed into a liquid; and the liquid fluorine-containing waste entered the liquid waste tank 14 for collection;
9: the remaining mixed gas entered the secondary condenser for condensation; the CF₄ gas was condensed into a liquid; and the liquid CF₄ entered the CF₄ collection tank 16 for collection; and
10: when it was required to stop the reaction, the sulfur hexafluoride gas cylinder 1 and the first pressure reducing valve 3 were closed; after ten minutes, the first-stage DBD plasma reactor 11, the second-stage DBD plasma reactor 12, the carbon dioxide gas cylinder 8, and the third pressure reducing valve 10 were closed, while the argon gas was continuously introduced to purge the mixed gas from the coaxial DBD reaction tubes of both the first-stage DBD plasma reactor 11 and the second-stage DBD plasma reactor 12; and after ten minutes, the primary condenser 13, the secondary condenser 15, the argon gas cylinder 4, and the second pressure reducing valve 6 were closed.

### Example 3

Compared with Example 2, the input power of the first-stage DBD plasma reactor was changed, and the output flow rate of the mixed gas was also changed to 150 mL/min; the concentration of SF₆ in the mixed gas discharged from the outlet of the first-stage DBD plasma reactor was detected by a gas chromatograph; and the degradation rate of SF₆ was calculated based on the concentration of SF₆ detected at the outlet of the first-stage DBD plasma reactor. The data graph showing the degradation rate of SF₆ changing with the input power of the first-stage DBD plasma reactor is shown in Table 1 below:

TABLE 1
Effect of the Input Power of the First-Stage DBD
Plasma Reactor on the Degradation Rate of SF₆

| | Degradation Rate of | |
|---|---|---|
| Input Power (W) | SF₆ (%) | Main Components |
| 60 | 61.7 | SO₂, SO₂F₂, SOF₂, |
| 70 | 73.4 | SOF₄, and SiF₄ |
| 80 | 80.2 | |
| 90 | 89.4 | |
| 100 | 99.8 | |

As can be seen from Table 1, when the input power of the first-stage DBD plasma reactor increased, the degradation rate of SF₆ also increased; when the input power of the first-stage DBD plasma reactor reached 100 W, the degradation rate of SF₆ reached 99.8%, approaching complete degradation.

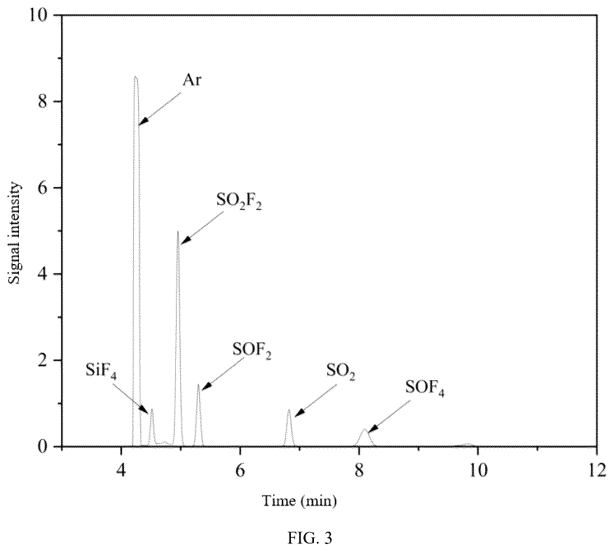

Furthermore, the mixed gas discharged from the outlet of the first-stage DBD plasma reactor was tested by a gas chromatograph. The obtained chromatogram is shown in FIG. 3. As can be seen from FIG. 3, the main components of the mixed gas discharged from the outlet of the first-stage DBD plasma reactor included SO₂, SO₂F₂, SOF₂, SOF₄, and SiF₄. These substances were presumed to be fluorine-containing small molecules obtained from the reaction of low-fluorine sulfides and fluorine atoms produced by the degradation of sulfur hexafluoride with silicon dioxide or oxygen atoms.

### Example 4

Compared with Example 2, the input power of the second-stage DBD plasma reactor was changed; the concentration of CF₄ in the mixed gas discharged from the outlet of the second-stage DBD plasma reactor was detected by a gas chromatograph-mass spectrometer; and the yield of CF₄ was calculated based on the concentration of CF₄ detected at the outlet of the second-stage DBD plasma reactor. The data graph showing the yield of CF₄ changing with the input power of the second-stage DBD plasma reactor is shown in Table 2 below:

TABLE 2
Effect of the Input Power of the Second-Stage
DBD Plasma Reactor on the Yield of CF₄

| Input Power (W) | Yield of CF₄ (%) |
|---|---|
| 60 | 7.6 |
| 70 | 14.7 |
| 80 | 19.3 |
| 90 | 25.1 |
| 100 | 29.4 |

As can be seen from Table 2, when the input power of the second-stage DBD plasma reactor increased, the yield of CF₄ also increased; when the input power of the second-stage DBD plasma reactor reached 100 W, the yield of CF₄ reached 29.4%, which is already considerable.

What is claimed is:

1. A method for synthesizing CF₄ by activating SF₆ using two-stage dielectric barrier discharge, comprising the following steps:
S1: introducing a background gas and a sulfur hexafluoride gas or sulfur hexafluoride waste gas into a first-stage dielectric barrier discharge plasma reactor, wherein the background gas is ionized to generate plasma, the sulfur hexafluoride, after being activated by the plasma, is ionized into fluorine atoms and low-fluorine sulfides, and the generated fluorine atoms and low-fluorine sulfides further react with oxygen and silicon dioxide to generate fluorine-containing small molecules; and
S2: subsequently introducing the fluorine-containing small molecules generated in the step S1 into a second-stage dielectric barrier discharge plasma reactor connected to the first-stage dielectric barrier discharge plasma reactor, and simultaneously introducing carbon dioxide gas into the second-stage dielectric barrier discharge plasma reactor, wherein the low-fluorine sulfides react with the carbon dioxide under an action of the plasma to generate carbon tetrafluoride gas.

2. The method for synthesizing the CF₄ by activating the SF₆ using the two-stage dielectric barrier discharge of claim 1, wherein the background gas is an inert gas.

3. The method for synthesizing the CF₄ by activating the SF₆ using the two-stage dielectric barrier discharge of claim 1, wherein in the step S1, a discharge region within the first-stage dielectric barrier discharge plasma reactor is filled with a catalyst γ-Al₂O₃.

4. The method for synthesizing the CF₄ by activating the SF₆ using the two-stage dielectric barrier discharge of claim 1, wherein the step S2 comprises: first performing primary condensation on a mixed gas containing carbon tetrafluoride flowing out from an outlet of the second-stage dielectric barrier discharge plasma reactor to condense fluorine-containing acidic waste gases into a liquid, then performing secondary condensation on the remaining mixed gas to condense the generated carbon tetrafluoride gas into a liquid, and collecting the liquid carbon tetrafluoride.

5. The method for synthesizing the CF₄ by activating the SF₆ using the two-stage dielectric barrier discharge of claim 4, wherein the primary condensation is performed at −60° C. to −50° C., and the secondary condensation is performed at −150° C. to −130° C.

6. An apparatus based on the method for synthesizing the CF₄ by activating the SF₆ using the two-stage dielectric barrier discharge of claim 1, comprising: a sulfur hexafluoride gas supply unit, a background gas supply unit, a first-stage dielectric barrier discharge plasma reactor, a second-stage dielectric barrier discharge plasma reactor, a carbon dioxide gas supply unit, a primary condensation unit, and a secondary condensation unit, wherein the sulfur hexafluoride gas supply unit and the background gas supply unit are respectively in communication with an inlet of the first-stage dielectric barrier discharge plasma reactor, an outlet of the first-stage dielectric barrier discharge plasma reactor and the carbon dioxide gas supply unit are respectively connected to an inlet of the second-stage dielectric barrier discharge plasma reactor, an outlet of the second-stage dielectric barrier discharge plasma reactor is connected to an inlet of the primary condensation unit, and a gas outlet of the primary condensation unit is connected to an inlet of the secondary condensation unit.

7. The apparatus for the method for synthesizing the CF₄ by activating the SF₆ using the two-stage dielectric barrier discharge of claim 6, further comprising: a gas mixer, wherein the sulfur hexafluoride gas supply unit comprises a sulfur hexafluoride gas cylinder, a first gas supply branch pipe, and a first pressure reducing valve, wherein an upstream end of the first gas supply branch pipe is connected to the sulfur hexafluoride gas cylinder, and the first pressure reducing valve is installed on the first gas supply branch pipe; the background gas supply unit comprises a background gas cylinder, a second gas supply branch pipe, and a second pressure reducing valve, wherein an upstream end of the second gas supply branch pipe is connected to the background gas cylinder, the second pressure reducing valve is installed on the second gas supply branch pipe, downstream ends of the first gas supply branch pipe and the second gas supply branch pipe are respectively connected to the gas mixer, and an outlet of the gas mixer is connected to the inlet of the first-stage dielectric barrier discharge plasma reactor.

8. The apparatus for the method for synthesizing the CF₄ by activating the SF₆ using the two-stage dielectric barrier discharge of claim 6, wherein the carbon dioxide gas supply unit comprises a carbon dioxide gas cylinder, a third gas supply branch pipe, and a third pressure reducing valve, wherein an upstream end of the third gas supply branch pipe is connected to the carbon dioxide gas cylinder, the third pressure reducing valve is installed on the third gas supply branch pipe, and a downstream end of the third gas supply branch pipe is connected to the inlet of the second-stage dielectric barrier discharge plasma reactor.

9. The apparatus for the method for synthesizing the CF₄ by activating the SF₆ using the two-stage dielectric barrier discharge of claim 6, wherein the primary condensation unit comprises a primary condenser, wherein the primary condenser comprises a first gas outlet and a first liquid outlet; the secondary condensation unit comprises a secondary condenser, wherein the secondary condenser comprises a second gas outlet and a second liquid outlet; the outlet of the second-stage dielectric barrier discharge plasma reactor is connected to an inlet of the primary condenser; and the first gas outlet is connected to an inlet of the secondary condenser.

10. The apparatus for the method for synthesizing the CF₄ by activating the SF₆ using the two-stage dielectric barrier discharge of claim 9, wherein the secondary condensation unit further comprises a CF₄ collection tank, and the second liquid outlet is connected to the CF₄ collection tank.

11. The apparatus for the method for synthesizing the CF₄ by activating the SF₆ using the two-stage dielectric barrier discharge of claim 9, wherein a condensing medium in the primary condenser is an ethylene glycol aqueous solution, and a condensing medium in the secondary condenser is liquid nitrogen.

12. The apparatus for the method for synthesizing the CF₄ by activating the SF₆ using the two-stage dielectric barrier discharge of claim 9, further comprising: an exhaust gas treatment unit, wherein the exhaust gas treatment unit comprises an alkali liquor treatment tank and an exhaust gas pipe, and both ends of the exhaust gas pipe are respectively connected to the alkali liquor treatment tank and the second gas outlet.
